(19) United States
(12) Patent Application Publication
(10) Pub. No.: US 2024/0105290 A1
(43) Pub. Date: Mar. 28, 2024

(54) TRACKING OBJECTS USING VIRTUAL IDENTIFIERS

(71) Applicants: Ionfield Holdings LLC, Moorestown, NJ (US); Computype Inc., St. Paul, MN (US)

(72) Inventors: David Lea Richmond, Pawleys Island, SC (US); Paul Fredric Hensley, Moorestown, NJ (US)

(73) Assignees: Ionfield Holdings LLC, Moorestown, NJ (US); Computype Inc., St. Paul, MN (US)

(21) Appl. No.: 18/471,886

(22) Filed: Sep. 21, 2023

Related U.S. Application Data
(60) Provisional application No. 63/409,384, filed on Sep. 23, 2022.

(51) Int. Cl. G16H 10/40; G01N 35/00

(52) U.S. Cl. CPC G16H 10/40; G01N 35/00732; G01N 2035/00752; G01N 2035/00782

(57) ABSTRACT

In some embodiments, a system tracks multiple uses of a physical object having a physical tracking tag with a physical identifier. The system reads the physical tracking tag to determine the physical identifier and assigns different, unique virtual identifiers for different uses of the object. The system may store each virtual identifier with information describing the corresponding use of the object. The system may generate a running cycle count for the object, where the system may use the cycle count to determine when to stop re-using the object. The object may be a refurbishable labware consumable, such as a physically barcoded microplate, where the system assigns virtual identifiers and cycle count to the consumable and cleans the consumable to enable multiple re-uses of the consumable with (i) each use being assigned a unique virtual identifier and (ii) the consumable having the same physical barcode.

### CROSS-REFERENCE TO RELATED APPLICATIONS

This application claims the benefit of the filing date of U.S. provisional application No. 63/409,384, filed on Sep. 23, 1922 and entitled “Virtual Barcode System”, the teachings of which are incorporated herein by reference in their entirety.

### BACKGROUND

### Field of the Disclosure

The present disclosure relates generally to tracking individual objects using unique identifiers, such as (without limitation) barcodes and RFID tags.

### Description of the Related Art

This section introduces aspects that may help facilitate a better understanding of the disclosure. Accordingly, the statements of this section are to be read in this light and are not to be understood as admissions about what is prior art or what is not prior art.

Traditionally, single-use labware consumables such as barcoded microplates are an essential product in the life science industry. The barcode or barcode identification information (i.e., the numerical value associated with and encoded in the barcode) associated with each microplate is specific and unique to each microplate and enables the user to scan, record, and track various information related to the microplate, including but not limited to date, testing conditions, information on the compounds or material supported in each well of the microplate being tested, and the type of assays that were run. After testing is complete, all the information related to the testing is saved, processed, and reviewed with the barcode identification information associated with the specifically tested microplate typically being the identifier of the testing information.

Every year, a typical laboratory consumes thousands of barcoded microplates, which are normally disposed of in landfills or burned after their single use, leading to significant environmental pollution and costs. Although the process of cleaning labware consumables is known in the art, the refurbishing (e.g., cleaning) of barcoded microplates presents difficulties due to the presence of the unique barcode identification information. That is, the use of a typical, refurbished barcoded microplate will result in the same barcode identification information being used for multiple separate tests which is why some laboratory software prevents the use of the same barcode identification information for different tests.

### SUMMARY

Thus, there is a need for a system or process of refurbishing of barcoded labware consumables, such as microplates, that will enable the re-use of the refurbished barcoded microplates without the potential of cross-over or a mix-up of previous testing information.

Briefly, according to certain embodiments, the present disclosure comprises a process for a combined virtual barcode and microplate recycler system that includes the steps of scanning a unique physical barcode located on a refurbishable microplate before the microplate is placed into service, assigning a unique virtual barcode and a cycle count (e.g., beginning at 0) to the microplate, placing the microplate into service to process samples in a laboratory setting, and storing test information referenced using the virtual barcode.

After the microplate is used, the system cleans the microplate, assigns a new unique virtual barcode to the refurbished microplate, and updates (e.g., increments by 1) the microplate's cycle count, where the microplate is then re-used to process another set of samples in the laboratory setting and store the new test information referenced using the new virtual barcode.

The system uses the virtual barcodes to uniquely record and track a digital recorded history of each use of the microplate, the number of re-uses of the microplate, the type of assays ran in each of the cycles (i.e., each use) of the microplate, the compounds supported in each well of the microplate in each cycle, information on total carbon savings by the number of re-uses of the microplate.

In some embodiments, the microplate is cleaned for another cycle of use in the laboratory setting by (i) running the microplate through a microplate cleaning system that utilizes a low-temperature atmospheric-pressure plasma process to destroy organic molecules residing in microplate wells to restore the microplate back to a pre-used condition after the microplate has been used for processing samples and then (ii) scanning the physical barcode to enable a microplate digital traceability platform located on a cloud server to recognize the unique physical barcode of the refurbished microplate, assign a new virtual barcode for the refurbished microplate, and increment the microplate's cycle count. The refurbished microplate is then returned to the laboratory to await re-use.

At some point in the process, the microplate may be inspected for re-use. The inspection process may include the steps of (i) removing the microplate from service when the microplate reaches an assigned maximum cycle count or if the microplate contains defects and (ii) replacing the microplate with a new microplate by up-loading the physical barcode of the new microplate to the digital traceability platform to assign a virtual barcode and an initial cycle count of 0 for the new microplate.

### BRIEF DESCRIPTION OF THE DRAWINGS

Embodiments of the disclosure will become more fully apparent from the following detailed description, the appended claims, and the accompanying drawings in which like reference numerals identify similar or identical elements.

FIG. 1 is a diagram representing the flow of processing according to one embodiment of the disclosure; and

FIG. 2 is a simplified block diagram of a generic (distributed or centralized) system for tracking physical objects according to certain embodiments of the present disclosure.

### DETAILED DESCRIPTION

Detailed illustrative embodiments of the present disclosure are disclosed herein. However, specific structural and functional details disclosed herein are merely representative for purposes of describing example embodiments of the present disclosure. The present disclosure may be embodied in many alternate forms and should not be construed as limited to only the embodiments set forth herein. Further, the terminology used herein is for the purpose of describing particular embodiments only and is not intended to be limiting of example embodiments of the disclosure.

As used herein, the singular forms “a,” “an,” and “the,” are intended to include the plural forms as well, unless the context clearly indicates otherwise. It further will be understood that the terms “comprises,” “comprising,” “contains,” “containing,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functions/acts involved.

The general purpose of certain embodiments of the present disclosure, which will be described subsequently in greater detail, is to provide a virtual tracking tag system for the recycling and/or monitoring of (i) physical objects that contain physical tracking tags that are either affixed to or embedded within the objects and (ii) their associated use such as, for example, in the life science industry.

There has thus been outlined the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

In this respect, before explaining at least one embodiment of the disclosure in detail, it is to be understood that the disclosure is not limited in its application to the details of construction and to the arrangements of the components set forth in the following description or illustrated in the drawings. The disclosure is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods, and systems for carrying out the several purposes of the present disclosure. It is important, therefore, that the disclosing subject matter be regarded as including such equivalent constructions insofar as they do not depart from the spirit and scope of the present disclosure.

The following detailed description is merely exemplary in nature and is not intended to limit the described embodiments of the disclosure and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention to be bound by any expressed or implied theory presented in the preceding technical field, background, brief summary, or the following detailed description.

In addition, the accompanying drawings, which are included to provide a further understanding of the disclosure are incorporated in and constitute a part of this specification, illustrate an embodiment of the disclosure and together with the description serve to explain the principles of the disclosure. They are meant to be exemplary illustrations provided to enable persons skilled in the art to practice the disclosure and are not intended to limit the scope of any potential claims.

According to certain embodiments of the present disclosure, a physical object contains a physical tracking tag containing or representing a unique physical identifier (PID) that enables the identification, authentication, and tracking of the physical object that the physical tracking tag is affixed to or embedded within. A tracking system assigns a different, unique virtual identifier (VID) to the physical object for each different use of the physical object. Although the physical tracking tag that is affixed to or embedded within the physical object and its physical identifier remain the same, the virtual identifier may change at the user's preference for each different use of the physical object. The primary function of the physical tracking tag in a tracking system of the present disclosure is to serve as an entry point to the tracking system. The virtual identifier may be overwritten with a new virtual identifier on each trigger event (e.g., completion of each use of the object).

The physical tracking tag may be either active or passive. Examples of suitable physical tracking tags include but are not limited to (i) passive, one- and two-dimensional optically readable codes, such as linear barcodes and 2-dimensional codes, and (ii) active, electronically readable tags, such as RFID (Radio-Frequency Identification) tags and NFC (Near Field Communication) tags, which are a subset of RFID tags. Although barcode tracking tags will be discussed below, other suitable types of physical tracking tags may readily replace the barcode tracking tags without changing the heart of the disclosure.

In general, a virtual barcode is a number that is associated with a physical barcode during one or multiple uses of a physical object affixed with the physical barcode. When a physical barcode is read, such as, for example, through a scanning process, the physical barcode is checked for being associated with a virtual barcode, and if so, the virtual barcode is used as a substitute or replacement for the physical barcode.

A trigger event happens when a physical object having a virtual barcode passes through a step in a process where the current virtual barcode is replaced with a new virtual barcode. From that step in the process, the new virtual barcode will be the substitute or replacement for the physical barcode until the next trigger event. Control of the virtual barcode process is by software that can be executed by a system processor on a local host network or called by an API (Application Programming Interface) over a distributed network or by another suitable process.

One embodiment of the present disclosure is directed to traditionally single-use labware consumables for the life science industry and includes (i) the use of a digital traceability platform for assigning virtual barcodes and (ii) the use of a cleaning system for refurbishing the labware consumables. The labware consumables may be any suitable type of refurbishable labware consumable such as (without limitation) microplates, pipettes, test tubes, molded tubes, beakers, Erlenmeyer flasks, filter flasks, vials, measuring cylinders, conical flasks, slides, reagent bottles, re-usable glassware, or other objects used as labware having a physical barcode with a PID for which a unique VID is a better confirmation of use in the lab process. Note that, although certain labware consumables, such as individual pipettes, might not have physical barcodes, the racks and other containment systems for batches of such labware that are used together often do. With the growing awareness of the need to minimize the use of single-use plastic labware, more uses of virtual barcodes will be developed.

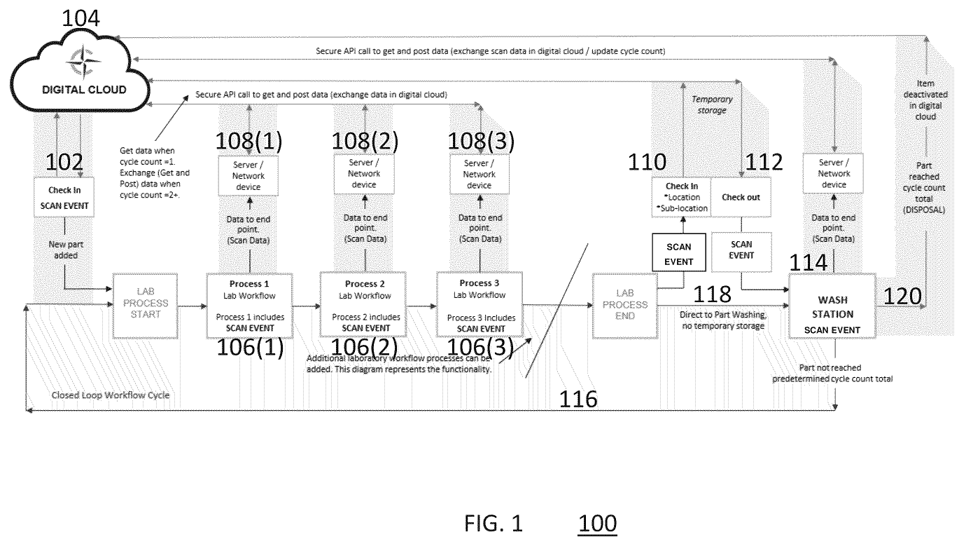

FIG. 1 is a diagram representing the flow of processing 100 according to one embodiment of the disclosure. The processing of FIG. 1 relates to the use, cleaning, and re-use of microplates in a laboratory (aka lab, for short), where each microplate has affixed to it a physical barcode that represents a unique physical identifier (i.e., the barcode number) for the microplate.

Referring to FIG. 1, when a new microplate is introduced into the lab, the microplate's physical barcode is scanned at a check-in station 102, which uploads the barcode's physical identifier (PID) for the new microplate to the digital cloud 104 (e.g., an example of the digital traceability platform referred to previously). In response, the digital cloud 104 assigns an initial virtual identifier (VID) (i.e., a virtual barcode number) to the microplate and stores in its memory a record containing the PID, the current VID, and an initial cycle count of 0 indicating that the microplate is going into its initial use.

The microplate is now ready for use in the lab. In this particular example, the lab workflow involves three different stages 106(1)-106(3) of lab work, labeled Process 1, Process 2, and Process 3, respectively. In general, lab work may involve any number of stages. At each stage 106, the microplate's physical barcode is scanned and the microplate's PID is uploaded to a corresponding server/network device 108, which transmits the PID to the digital cloud 104. In response, the digital cloud 104 retrieves the corresponding VID from its memory and transmits the VID back to the corresponding server/network device 108, which links the VID to the testing results generated at the corresponding workflow stage 106. Depending on the particular implementation, the multiple instances of the server/network device 108(1)-108(3) shown in FIG. 1 may be distributed devices that communicate with one another or a single centralized server.

After all of the different stages 106 of the lab workflow are completed, the microplate may be temporarily stored, for example, to accumulate a number of microplates for cleaning. The temporary storage phase may involve scanning the microplate's physical barcode at a check-in station 110 and uploading the microplate's PID to the digital cloud 104, which updates its record for the microplate to indicate that the microplate has completed the current workflow process and is entering temporary storage.

After a suitable number of microplates have been accumulated into temporary storage, the accumulated set of microplates is then ready to be washed. At that point, the physical barcode of each microplate may be scanned at a check-out station 112, and the corresponding PID is uploaded to the digital cloud 104, which updates each corresponding record to indicate that the microplate is leaving temporary storage.

At the wash station 114, the physical barcode of each microplate is again scanned and transmitted to the digital cloud 104 along with an indication that a trigger event has occurred (i.e., that the previous lab workflow has been completed). In response to receiving the PID and the trigger event indication, the digital cloud 104 assigns a new VID for that PID in its memory and increments the corresponding cycle count n to indicate that the microplate is to be available to be used for the (n+1)th time in the lab. Note that, in other implementations, the trigger event could occur at any point after the previous lab workflow has been completed, such as as the microplate is being check into or out of temporary storage. After the microplate has been cleaned at the wash station 114, it is available to be re-used for another instance of (the same or different) lab workflow process, as indicated in FIG. 1 at flow line 116.

Note that, in an alternative implementation, the microplate bypasses the temporary storage phase and, instead, proceeds directly to the wash station 114 at the completion of each lab workflow, as indicated in FIG. 1 at flow line 118.

At some point in the processing 100, the digital cloud 104 is interrogated using the microplate's PID to retrieve the microplate's current cycle count. If and when the microplate's current cycle count reaches a specified maximum number of uses for a microplate (if any), the microplate is removed from the lab and discarded, as indicated in FIG. 1 at flow line 120. In FIG. 1, this determination is indicated as being made at the wash station 114. In other implementations, this determination may be made at any point in the process after the previous lab workflow has been completed and before the next lab workflow is initiated.

In addition to removing a microplate from further re-use based on its cycle count reaching the specified maximum number of uses, a microplate may also be removed based on defects in the microplate itself. Depending on the implementation, these defects may be detected automatically by machinery or manually by technicians at any suitable point in the processing 100 of FIG. 1. If and when a microplate is removed, in some implementations not represented in FIG. 1, the microplate's physical barcode is scanned and the microplate's PID is uploaded to the digital cloud 104 with an indication that the microplate is being removed from service prematurely (i.e., before its cycle count reaches the specified maximum number of uses).

The information stored in the digital cloud 104 regarding (i) the cycle count for each microplate currently in service and (ii) the received indications of microplate removals can be used to maintain an adequate inventory of microplates for the lab. In particular, knowledge of (i) how many microplates are currently in service and (ii) how many more uses are potentially available for each of those microplates can be used to determine when to order more microplates from a supplier.

By linking the lab results from each stage 106 of the same instance of the lab workflow to the same virtual identifier, all of those lab results from that lab workflow instance will be available for analysis together. By assigning a different, unique virtual identifier for each different use of each different microplate, the different sets of lab results can be analyzed without confusing different uses of even the same microplate.

In some implementations, a virtual identifier may be transferred from one microplate to another microplate. For example, referring to FIG. 1, for a given instance of the lab workflow, samples may be transferred from a current microplate to a new microplate, such as between stages 106(1) and 106(2) or between stages 106(2) and 106(3). In that case, the digital cloud 104 will already have an existing record that links the physical identifier for the current microplate to that microplate's current virtual identifier. In order to transfer that virtual identifier to the new microplate, the physical barcode for the current microplate is scanned and the corresponding physical identifier is uploaded to the digital cloud 104 along with an indication that the associated virtual identifier is to be transferred to a different microplate. The physical barcode for the new microplate is then scanned and the corresponding physical identifier is uploaded to the digital cloud 104, which will then create a new record for the new microplate linking the existing virtual identifier to the new microplate's physical identifier so that all of the lab results for that same lab workflow will be associated with the same virtual identifier. Note that the digital cloud 104 will set the cycle count for the new microplate to 0 if the current lab workflow is the first use of the new microplate. If the new microplate is a refurbished microplate that has been used before, then the refurbished microplate will already have a record in the digital cloud 104, in which case, its cycle count will need to be incremented from its previous value.

There are many different ways in which VIDs may be assigned to microplates. For most applications, the primary criterion is that each VID be unique so that the lab results for each use of each microplate will be uniquely identifiable. In some implementations, the VIDs may be randomly generated as long as they are unique. In some implementations, the length (i.e., the number of characters) of the VIDs needs to be no longer than the length of the PIDs. In some implementations, different ranges of VID values are reserved for different types of lab work. In some implementations, each VID is generated by appending additional characters to the corresponding PID. For example, the additional characters may be the cycle count that indicates the particular use of the microplate.

Some advantages of the present disclosure may include that business decisions and processes can move faster, oversight is reduced, duplication of effort is greatly reduced or eliminated, centralized transactions and information can be integrated into an organization's existing systems and processes, the ability to track physical assets and data flexibly, comprehensively, and securely, providing near real-time visibility into customized fields such as location, status, expiration date, and environmental conditions for all of one's assets, providing complete chain-of-custody records with immutable data, the ability to easily integrate other data inputs/outputs into the platform, providing users with increased efficiency, lower cost, and reduced risk, and providing a complete, end-to-end solution for track-and-trace monitoring and inventory management that integrates into existing processes and systems.

In some implementations of the processing 100 of FIG. 1, the wash station is the PurePLATE™ Microplate Cleaning System (MCS) by IonField Systems (IFS) of Moorestown, New Jersey, which utilizes a low-temperature, atmospheric-pressure plasma process to destroy organic molecules residing in microplate wells, thereby enabling microplates to be repeatedly re-used. Microplates are ready for immediate re-use and there is no known limit to the number of re-uses for a majority of the standard SBS (Society for Biomolecular Screening) microplates.

Some of the features of the PurePLATE™ Microplate Cleaning System include but are not limited to the ability to clean microplates with a standard SBS footprint of up to 44 mm, the ability to process up to 50-55 plates per hour, providing relatively inexpensive processing costs compared to buying new plates, providing laboratories with the opportunity to actively reduce their greenhouse gas emissions, such as by over 50% over the prior art, providing intuitive software having the ability to adapt to specific cleaning requirements, providing for modular and self-monitoring components to simplify maintenance, and providing quick start-ups including, for example, being powered up, primed, and operational in under 5 minutes.

A feature of certain embodiments of the present disclosure is that the ability to clean and re-use barcoded microplates provides the user with the ability to identify and isolate microplate-related noise and account for these noises in the future uses of the microplate to provide for more-accurate testing using and re-using the same refurbished microplate compared to the use of a different, new microplate for each different test.

Use of each of the microplates may include but is not limited to storage of a digitally recorded history detailing information regarding the microplate such as but not limited to each usage of the microplate, the number of re-uses of the microplate, what assays were run in each of the cycles of the microplate, what compounds were supported in each well of the microplate in each cycle, information on total carbon savings by the number of re-uses of the microplate such as, for example, based on microplate type, and numerous other features, including but not limited to using standard tools such as artificial intelligence employed to make the software package provide more utility and value to the user.

Although the disclosure has been described in the context of a system that cleans refurbishable labware consumables such as barcoded microplates and assigns a new virtual barcode for each use of each barcoded microplate, the disclosure is not so limited. In general, the disclosure may be implemented in any suitable system that tracks physical objects having physical tracking tags having unique physical identifiers by assigning a different, unique virtual identifier for each different subset of one or more uses of the object. In this way, the object may be re-used multiple times with each subset of uses being assigned its own unique virtual identifier even though the object has only one physical identifier for all of the uses of that object.

In some implementations, each subset of uses has only a single use such that each use of the object is assigned its own unique virtual identifier. In that case, since the object has a physical identifier that uniquely distinguishes it from other objects, in some implementations, the virtual identifiers for a given object can be generated by appending a unique cycle count to the object's physical identifier. As a result, the virtual identifiers will uniquely distinguish both (i) each use of the object from all other uses of that same object and (ii) all of the uses of the object from all of the uses of all other objects.

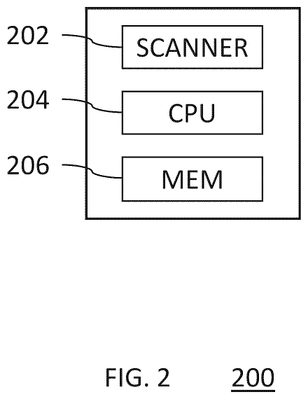

FIG. 2 is a simplified block diagram of a generic (distributed or centralized) system 200 for tracking physical objects according to certain embodiments of the present disclosure. As shown in FIG. 2, the system 200 includes (i) a scanner (e.g., barcode reader, RFID reader)) 202 that scans (e.g., reads or interrogates) physical tracking tags on the physical objects, (ii) a processor (e.g., CPU microprocessor) 204 that controls the operations of the system 200, and (iii) a memory (e.g., RAM, ROM) 206 that stores code executed by the processor 204 and/or data generated and/or received by the system 200. In some implementations, for each physical object having a physical identifier, the processor 204 (i) generates and tracks the current virtual identifier and a running cycle count for each use of the physical object and (ii) stores, in the memory 206, information (e.g., lab test results) for each use along with the physical identifier, the virtual identifier, and the cycle count of that use.

In certain embodiments, the present disclosure is a machine-implemented method for tracking multiple uses of a physical object having a physical tracking tag with a physical identifier. The method comprises reading the physical tracking tag to determine the physical identifier; assigning, to the object, a first virtual identifier for a first subset of one or more uses of the object; and assigning, to the object, a second virtual identifier for a second subset of one or more uses of the object, wherein the second virtual identifier is different from the first virtual identifier.

In at least some of the above embodiments, the first and second subsets each correspond to a different, single use of the object.

In at least some of the above embodiments, the method further comprises generating a cycle count for the object, wherein the cycle count is incremented for each consecutive use of the object.

In at least some of the above embodiments, the method further comprises performing a comparison between the cycle count and a specified maximum cycle count; and determining whether to terminate re-use of the object based on the comparison.

In at least some of the above embodiments, each virtual identifier is generated by appending together the physical identifier and the corresponding cycle count.

In at least some of the above embodiments, the method further comprises storing in memory each virtual identifier along with information describing the corresponding use of the object.

In at least some of the above embodiments, the method further comprises determining whether the object is suitable for re-use independent of the virtual identifier.

In at least some of the above embodiments, the method further comprises transferring a current virtual identifier for the object to a different object.

In at least some of the above embodiments, the object is a refurbishable labware consumable; and the method involves a system for cleaning the refurbishable labware consumable such that the refurbishable labware consumable is re-used multiple times with (i) each use having a unique virtual identifier and (ii) the refurbishable labware consumable having the same physical identifier.

In at least some of the above embodiments, the method further comprises assigning and storing, in a memory, a different virtual identifier and an incrementing cycle count for each different use of the refurbishable labware consumable; and retrieving, from the memory, a current virtual identifier and a current cycle count in response to receiving the physical identifier of the refurbishable labware consumable.

In at least some of the above embodiments, the method further comprises using the refurbishable labware consumable at a plurality of stages of lab workflow; reading the physical tracking tag to determine the physical identifier at each stage; retrieving, from the memory, the current virtual identifier based on the physical identifier; and linking lab results from each stage to the current virtual identifier.

In certain embodiments, the present disclosure is a system for tracking multiple uses of a physical object having a physical tracking tag with a physical identifier. The system comprises at least one scanner configured to read the physical tracking tag to determine the physical identifier; and at least one processor configured to assign, to the object, a first virtual tracking tag having a first virtual identifier for a first subset of one or more uses of the object; and assign, to the object, a second virtual tracking tag having a second virtual identifier for a second subset of one or more uses of the object, wherein the second virtual identifier of the second virtual tracking tag is different from the first virtual identifier of the first virtual tracking tag.

In at least some of the above embodiments, the first and second subsets each correspond to a different, single use of the object.

In at least some of the above embodiments, the at least one processor is configured to generate a cycle count for the object, wherein the cycle count is incremented for each consecutive use of the object.

In at least some of the above embodiments, the at least one processor is configured to perform a comparison between the cycle count and a specified maximum cycle count; and determine whether to terminate re-use of the object based on the comparison.

In at least some of the above embodiments, the at least one processor is configured to generate each virtual identifier by appending together the physical identifier and the corresponding cycle count.

In at least some of the above embodiments, the system further comprises at least one memory configured to store each virtual identifier along with information describing the corresponding use of the object.

In at least some of the above embodiments, the at least one processor is configured to determine whether the object is suitable for re-use independent of the virtual identifier.

In at least some of the above embodiments, the at least one processor is configured to transfer a current virtual identifier for the object to a different object.

In at least some of the above embodiments, the object is a refurbishable labware consumable; and the system comprises a wash station configured to clean the refurbishable labware consumable such that the refurbishable labware consumable is re-usable multiple times with (i) each use having a unique virtual identifier and (ii) the refurbishable labware consumable having the same physical identifier.

In at least some of the above embodiments, the system further comprises a digital traceability platform configured to assign and store, in a memory, a different virtual identifier and an incrementing cycle count for each different use of the refurbishable labware consumable; and retrieve, from the memory, a current virtual identifier and a current cycle count in response to receiving the physical identifier of the refurbishable labware consumable.

The functions of the various elements shown in the figures, including any functional blocks labeled as “processors” and/or “controllers,” may be provided through the use of dedicated hardware as well as hardware capable of executing software in association with appropriate software. Upon being provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non-volatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

As will be appreciated by one of ordinary skill in the art, the present disclosure may be embodied as an apparatus (including, for example, a system, a network, a machine, a device, a computer program product, and/or the like), as a method (including, for example, a business process, a computer-implemented process, and/or the like), or as any combination of the foregoing. Accordingly, embodiments of the present disclosure may take the form of an entirely software-based embodiment (including firmware, resident software, micro-code, and the like), an entirely hardware embodiment, or an embodiment combining software and hardware aspects that may generally be referred to herein as a “system” or “network”.

Embodiments of the disclosure can be manifest in the form of methods and apparatuses for practicing those methods. Embodiments of the disclosure can also be manifest in the form of program code embodied in tangible media, such as magnetic recording media, optical recording media, solid state memory, floppy diskettes, CD-ROMs, hard drives, or any other non-transitory machine-readable storage medium, wherein, upon the program code being loaded into and executed by a machine, such as a computer, the machine becomes an apparatus for practicing the disclosure. Embodiments of the disclosure can also be manifest in the form of program code, for example, stored in a non-transitory machine-readable storage medium including being loaded into and/or executed by a machine, wherein, upon the program code being loaded into and executed by a machine, such as a computer, the machine becomes an apparatus for practicing the disclosure. Upon being implemented on a general-purpose processor, the program code segments combine with the processor to provide a unique device that operates analogously to specific logic circuits. The term “non-transitory,” as used herein, is a limitation of the medium itself (i.e., tangible, not a signal) as opposed to a limitation on data storage persistency (e.g., RAM vs. ROM).

What is claimed is:

1. A machine-implemented method for tracking multiple uses of a physical object having a physical tracking tag with a physical identifier, the method comprising:
reading the physical tracking tag to determine the physical identifier;
assigning, to the object, a first virtual identifier for a first subset of one or more uses of the object; and
assigning, to the object, a second virtual identifier for a second subset of one or more uses of the object, wherein the second virtual identifier is different from the first virtual identifier.

2. The method of claim 1, wherein the first and second subsets each correspond to a different, single use of the object.

3. The method of claim 1, further comprising generating a cycle count for the object, wherein the cycle count is incremented for each consecutive use of the object.

4. The method of claim 3, further comprising:
performing a comparison between the cycle count and a specified maximum cycle count; and
determining whether to terminate re-use of the object based on the comparison.

5. The method of claim 3, wherein each virtual identifier is generated by appending together the physical identifier and the corresponding cycle count.

6. The method of claim 1, further comprising storing in memory each virtual identifier along with information describing the corresponding use of the object.

7. The method of claim 1, further comprising determining whether the object is suitable for re-use independent of the virtual identifier.

8. The method of claim 1, further comprising transferring a current virtual identifier for the object to a different object.

9. The method of claim 1, wherein:
the object is a refurbishable labware consumable; and
the method involves a system for cleaning the refurbishable labware consumable such that the refurbishable labware consumable is re-used multiple times with (i) each use having a unique virtual identifier and (ii) the refurbishable labware consumable having the same physical identifier.

10. The method of claim 9, further comprising:
assigning and storing, in a memory, a different virtual identifier and an incrementing cycle count for each different use of the refurbishable labware consumable; and
retrieving, from the memory, a current virtual identifier and a current cycle count in response to receiving the physical identifier of the refurbishable labware consumable.

11. The method of claim 10, further comprising:
using the refurbishable labware consumable at a plurality of stages of lab workflow;
reading the physical tracking tag to determine the physical identifier at each stage;
retrieving, from the memory, the current virtual identifier based on the physical identifier; and linking lab results from each stage to the current virtual identifier.

12. A system for tracking multiple uses of a physical object having a physical tracking tag with a physical identifier, the system comprising:
at least one scanner configured to read the physical tracking tag to determine the physical identifier; and
at least one processor configured to:
assign, to the object, a first virtual tracking tag having a first virtual identifier for a first subset of one or more uses of the object; and
assign, to the object, a second virtual tracking tag having a second virtual identifier for a second subset of one or more uses of the object, wherein the second virtual identifier of the second virtual tracking tag is different from the first virtual identifier of the first virtual tracking tag.

13. The system of claim 12, wherein the first and second subsets each correspond to a different, single use of the object.

14. The system of claim 12, wherein the at least one processor is configured to generate a cycle count for the object, wherein the cycle count is incremented for each consecutive use of the object.

15. The system of claim 14, wherein the at least one processor is configured to:
perform a comparison between the cycle count and a specified maximum cycle count; and
determine whether to terminate re-use of the object based on the comparison.

16. The system of claim 14, wherein the at least one processor is configured to generate each virtual identifier by appending together the physical identifier and the corresponding cycle count.

17. The system of claim 12, further comprising at least one memory configured to store each virtual identifier along with information describing the corresponding use of the object.

18. The system of claim 12, wherein the at least one processor is configured to determine whether the object is suitable for re-use independent of the virtual identifier.

19. The system of claim 12, wherein the at least one processor is configured to transfer a current virtual identifier for the object to a different object.

20. The system of claim 12, wherein:
the object is a refurbishable labware consumable; and
the system comprises a wash station configured to clean the refurbishable labware consumable such that the refurbishable labware consumable is re-usable multiple times with (i) each use having a unique virtual identifier and (ii) the refurbishable labware consumable having the same physical identifier.

21. The system of claim 20, further comprising a digital traceability platform configured to:
assign and store, in a memory, a different virtual identifier and an incrementing cycle count for each different use of the refurbishable labware consumable; and
retrieve, from the memory, a current virtual identifier and a current cycle count in response to receiving the physical identifier of the refurbishable labware consumable.
